(19) United States
(12) Patent Application Publication
(10) Pub. No.: US 2026/0160754 A1
(43) Pub. Date: Jun. 11, 2026

(54) UMBILICAL LINING-DERIVED STEM CELLS FOR TREATING IDIOPATHIC INFLAMMATION

(71) Applicant: RESTEM Group, Inc., Corona, CA (US)

(72) Inventors: Rafael Gonzalez, Corona, CA (US); Keith March, Corona, CA (US)

(21) Appl. No.: 19/344,324

(22) Filed: Sep. 29, 2025

Related U.S. Application Data
(63) Continuation-in-part of application No. 19/264,712, filed on Jul. 9, 2025.
(60) Provisional application No. 63/669,017, filed on Jul. 9, 2024.

(51) Int. Cl. G01N 33/50

(52) U.S. Cl. CPC G01N 33/5023; G01N 33/5047; G01N 2800/52

(57) ABSTRACT

A method of diagnosing and treating idiopathic inflammation in an individual is described. The method involves examining blood for inflammation factors in vitro and further involves administering an effective amount of umbilical lining-derived stem cells to the individual. The method may involve reducing or tapering steroid usage in individual afflicted with idiopathic inflammatory myopathy or other idiopathic inflammatory conditions. The method may involve administering a single dose of the umbilical lining-derived stem cells, or administering multiple doses of the umbilical lining-derived stem cells over a defined period of time.

### CROSS-REFERENCE TO RELATED APPLICATIONS

This continuation in part application claims priority to application Ser. No. 17/246,712 filed on Jul. 9, 2025 which claims priority to provisional application No. 63/669,017 filed on Jul. 9, 2024 entitled “Umbilical Lining-Derived Stem Cells For Treating Idiopathic Inflammatory Myopathy,” which is entirely incorporated by reference for all purposes.

### FIELD

This disclosure relates generally to clinical evaluation of human blood samples and mesenchymal stem cell (MSC) cellular therapy. More specifically, this disclosure is related to improved methods for blood detection of inflammation and use of MSCs isolated from the umbilical cord or Umbilical Cord Modulation Progenitor Cells (UCMPCs) for treatment of a broad range of inflammatory conditions.

### BACKGROUND

Idiopathic Inflammation (II) is inflammation resulting from an unknown etiology. Dermatomyositis (DM) and polymyositis (PM) are representative examples of resulting disorders characterized by proximal muscle weakness, and by skin involvement in DM. They have a significant adverse impact on quality of life, and are frequently associated with life threatening complications such as interstitial lung disease (ILD) (Connors, G. R., et al., Chest, 2010. 138 (6): p. 1464-74), neoplasia (Qiang, J. K., et al., J Cutan Med Surg, 2017. 21(2): p. 131-136) and cardiac disease (Zhang, L., et al., Clin Cardiol, 2012. 35(11): p. 686-91).

Patients with II frequently require years of treatment with powerful immunosuppressive drugs such as methotrexate, azathioprine and mycophenolate mofetil, which are associated with significant side-effects, ranging from infertility to neoplasia (Brewer, J. D., et al., Arch Dermatol, 2009. 145(12): p. 1391-6.; Pasternak, B., et al., American Journal of Epidemiology, 2013. 177 (11): p. 1296-1305). Other clinical examples of II may include and are not limited to: inclusion body myositis, idiopathic juvenile arthritis, idiopathic spondylarthrite, idiopathic transverse myelitis, idiopathic optic neuritis, idiopathic intracranial hypertension, idiopathic pulmonary fibrosis (IPF), idiopathic interstitial pneumonias (umbrella group with several subtypes), cryptogenic organizing pneumonia (idiopathic form of bronchiolitis obliterans organizing pneumonia), idiopathic inflammatory bowel disease (IBD) including Crohn's disease, and ulcerative colitis, idiopathic granulomatous hepatitis, idiopathic urticaria (chronic spontaneous urticaria); idiopathic panniculitis; idiopathic vasculitis, idiopathic crescentic glomerulonephritis; idiopathic nephrotic syndrome, idiopathic pericarditis, idiopathic myocarditis, idiopathic aortitis, idiopathic systemic vasculitides. Novel diagnostic tools and therapies with an improved risk/benefit profile are needed for treatment of idiopathic inflammatory disorders.

Dermatomyositis (DM) and polymyositis (PM) are representative idiopathic inflammatory myopathy disorders characterized by proximal muscle weakness, and by skin involvement in DM. They have a significant adverse impact on quality of life, and are frequently associated with life-threatening complications such as interstitial lung disease (ILD) (Connors, G. R., et al., Chest, 2010. 138(6): p. 1464-74), neoplasia (Qiang, J. K., et al., J Cutan Med Surg, 2017. 21(2): p. 131-136) and cardiac disease (Zhang, L., et al., Clin Cardiol, 2012. 35(11): p. 686-91).

Patients with DM and PM frequently require years of (or, prolonged) treatment with powerful immunosuppressive drugs such as methotrexate, azathioprine and mycophenolate mofetil, which are associated with significant side-effects, ranging from infertility to neoplasia (Brewer, J. D., et al., Arch Dermatol, 2009. 145(12): p. 1391-6.; Pasternak, B., et al., American Journal of Epidemiology, 2013. 177(11): p. 1296-1305). Novel therapies with an improved risk/benefit profile are needed for idiopathic inflammatory myopathy disorders, including but not limited to DM and PM.

Mesenchymal stem cells, also known as a mesenchymal stromal cells, are a strategic cell type in translational medicine because they have proven relatively safe in clinical studies, may be obtained from non-embryonic sources, and have great therapeutic potential. MSCs were first discovered in bone marrow (BM) and this continues to be a primary source for the cells. (Friedenstein A J, et al., The Development of Fibroblast Colonies in Monolayer Cultures of Guinea-Pig Bone Marrow and Spleen Cells. Cell Tissue Kinet. 1970; 3:393-403). However because a sample from the inner bone is required to derive these MSCs, other less invasive sources of MSCs have been located in fat as well as in cartilage, muscle and teeth. An even less invasive source of MSCs is the umbilical cord. Umbilical cord derived MSCs have great therapeutic potential because of their neonatal source.

MSCs from multiple tissues have been studied in treatment of bone fractures, osteoarthritis and rheumatoid arthritis, graft versus host disease, Crohn's disease, amyotrophic lateral sclerosis, and myocardial infarction, ischemic stroke, liver and kidney failure to name a few (Levy, O., et al, Shattering barriers toward clinically meaningful MSC therapies. Sci. Adv. 2020; eaba6884 22 Jul. 2020). Much of this work relied upon systemic infusion of bone marrow derived cells. To date, MSCs have shown the greatest clinical potential in treatment of conditions related to inflammation because of their immunomodulatory characteristics.

MSCs are formally defined by standardized criteria: adherent in cell culture; express markers CD90, CD73, and CD105; test negative for hematopoietic markers CD34, CD45, markers for monocytes, macrophages, and lymphocytes; and able to differentiate in vitro into osteoblasts, adipocytes and chondroblasts under standard culture conditions (Dominici et al., Minimal criteria for defining multi-potent mesenchymal stromal cells. The international society for cellular therapy position statement. Cytotherapy. 2006; 8 (4): 315-317). Despite these clear criteria for MSC identification, translational applications of MSCs remain limited by inconsistent reproducibility. One reason for this inconsistency is the considerable heterogeneity in the MSCs and procedures that have been tested including variation in the age and health of MSC donors, tissue source, method of isolation, method of cryopreservation, method of cell culture and method of scale up to clinically relevant population sizes, as well as in potency and quality assays. Attempts at standardizing or improving MSCs by genetic modification, derivation from embryonic cells and through reprogramming further add to the heterogeneity of MSCs under study.

### SUMMARY

For purposes of summarizing the disclosure and the advantages achieved over the prior art, certain objects and advantages of the disclosure are described herein. Not all such objects or advantages may be achieved in any particular embodiment. Thus for example, those skilled in the art will recognize that the invention may be embodied or carried out in a manner that achieves or optimizes one advantage or group of advantages as taught herein without necessarily achieving the other objects or advantages has may be taught or suggested herein.

In an aspect, a method of treating idiopathic inflammation in an individual is disclosed. The method involves administering an effective amount of ULSCs to the individual in need thereof.

In an aspect, a method of treating idiopathic inflammatory myopathy in an individual is disclosed. The method involves administering an effective amount of umbilical lining-derived stem cells to the individual in need thereof.

In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of muscle weakness and autoantibodies, including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM).

In embodiments, the idiopathic inflammatory conditions may comprise polymyositis (PM), dermatomyositis (DM) (including juvenile, amyopathic, and sine-dermatitis form), inclusion-body myositis (IBM), immune-mediated necrotizing myopathy (IMNM), focal autoimmune myositis, Lambert-Eaton myasthenic syndrome, myasthenia gravis, the muscle form of sarcoidosis, idiopathic juvenile arthritis, idiopathic spondylarthrites, rheumatoid arthritis, psoriatic arthritis, LUPUS, idiopathic transverse myelitis, idiopathic optic neuritis, idiopathic intracranial hypertension, idiopathic pulmonary fibrosis (IPF), idiopathic interstitial pneumonias (umbrella group with several subtypes), cryptogenic organizing pneumonia (idiopathic form of bronchiolitis obliterans organizing pneumonia), idiopathic inflammatory bowel disease (IBD) including Crohn's disease, and ulcerative colitis, idiopathic granulomatous hepatitis, idiopathic urticaria (chronic spontaneous urticaria); idiopathic panniculitis; idiopathic vasculitis, idiopathic crescentic glomerulonephritis; idiopathic nephrotic syndrome, idiopathic pericarditis, idiopathic myocarditis, idiopathic aortitis, idiopathic systemic vasculitis's. In certain embodiments, idiopathic inflammation refers to any one or combination of conditions recited herein.

In another aspect a method is disclosed for identification of patients with inflammatory conditions that would otherwise remain undetected in a standard blood plasma assays such as C-Reactive Protein (CRP) and Erythrocyte Sedimentation rate (ESR).

In embodiments, the effective amount of the umbilical lining-derived stem cells is at least about 50 million cells. In embodiments, the effective amount of the umbilical lining-derived stem cells is at least about 100 million cells. In embodiments, the effective amount of the umbilical lining-derived stem cells is at least about 200 million cells. In embodiments, the administering involves administering a single dose of the umbilical lining-derived stem cells. In other embodiments, the administering involves administering multiple doses of the umbilical lining-derived stem cells over a defined period of time.

In another aspect, a method is disclosed for reducing steroid usage or treatment in an individual afflicted or otherwise diagnosed with idiopathic inflammatory myopathy. The method involves administering an effective amount of umbilical lining-derived stem cells to the individual in need thereof.

In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of muscle weakness and autoantibodies, including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM).

In embodiments, the idiopathic inflammatory conditions may comprise polymyositis (PM), dermatomyositis (DM) (including juvenile, amyopathic, and sine-dermatitis form), inclusion-body myositis (IBM), immune-mediated necrotizing myopathy (IMNM), focal autoimmune myositis, Lambert-Eaton myasthenic syndrome, myasthenia gravis, the muscle form of sarcoidosis, idiopathic juvenile arthritis, idiopathic spondylarthrites, rheumatoid arthritis, psoriatic arthritis, LUPUS, idiopathic transverse myelitis, idiopathic optic neuritis, idiopathic intracranial hypertension, idiopathic pulmonary fibrosis (IPF), idiopathic interstitial pneumonias (umbrella group with several subtypes), cryptogenic organizing pneumonia (idiopathic form of bronchiolitis obliterans organizing pneumonia), idiopathic inflammatory bowel disease (IBD) including Crohn's disease, and ulcerative colitis, idiopathic granulomatous hepatitis, idiopathic urticaria (chronic spontaneous urticaria); idiopathic panniculitis; idiopathic vasculitis, idiopathic crescentic glomerulonephritis; idiopathic nephrotic syndrome, idiopathic pericarditis, idiopathic myocarditis, idiopathic aortitis, idiopathic systemic vasculitis's. In certain embodiments, idiopathic inflammation refers to any one or combination of conditions recited herein.

In another aspect a method is disclosed for identification of patients with inflammatory conditions that would otherwise remain undetected in a standard blood plasma assays such as C-Reactive Protein (CRP) and Erythrocyte Sedimentation rate (ESR).

In embodiments, the steroid comprises prednisone, prednisolone, methyl prednisone, dexamethasone, betamethasone, or triamcinolone, or derivates thereof. In certain embodiments, the steroid comprises prednisone.

In embodiments, the effective amount of the umbilical lining-derived stem cells is at least about 50 million cells. In embodiments, the effective amount of the umbilical lining-derived stem cells is at least about 100 million cells. In embodiments, the effective amount of the umbilical lining-derived stem cells is at least about 200 million cells. In embodiments, the administering involves administering a single dose of the umbilical lining-derived stem cells. In other embodiments, the administering involves administering multiple doses of the umbilical lining-derived stem cells over a defined period of time.

In another aspect, a method of tapering steroid usage in an individual afflicted with idiopathic inflammatory myopathy is disclosed. The method involves administering an effective amount of umbilical lining-derived stem cells to the individual in need thereof.

In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of muscle weakness and autoantibodies, including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM).

In embodiments, the idiopathic inflammatory conditions may comprise polymyositis (PM), dermatomyositis (DM) (including juvenile, amyopathic, and sine-dermatitis form), inclusion-body myositis (IBM), immune-mediated necrotizing myopathy (IMNM), focal autoimmune myositis, Lambert-Eaton myasthenic syndrome, myasthenia gravis, the muscle form of sarcoidosis, idiopathic juvenile arthritis, idiopathic spondylarthrites, rheumatoid arthritis, psoriatic arthritis, LUPUS, idiopathic transverse myelitis, idiopathic optic neuritis, idiopathic intracranial hypertension, idiopathic pulmonary fibrosis (IPF), idiopathic interstitial pneumonias (umbrella group with several subtypes), cryptogenic organizing pneumonia (idiopathic form of bronchiolitis obliterans organizing pneumonia), idiopathic inflammatory bowel disease (IBD) including Crohn's disease, and ulcerative colitis, idiopathic granulomatous hepatitis, idiopathic urticaria (chronic spontaneous urticaria); idiopathic panniculitis; idiopathic vasculitis, idiopathic crescentic glomerulonephritis; idiopathic nephrotic syndrome, idiopathic pericarditis, idiopathic myocarditis, idiopathic aortitis, idiopathic systemic vasculitis's. In certain embodiments, idiopathic inflammation refers to any one or combination of conditions recited herein.

In another aspect a method is disclosed for identification of patients with inflammatory conditions that would otherwise remain undetected in a standard blood plasma assays such as C-Reactive Protein (CRP) and Erythrocyte Sedimentation rate (ESR).

In embodiments, the steroid comprises prednisone, prednisolone, methyl prednisone, dexamethasone, betamethasone, or triamcinolone, or derivates thereof. In certain embodiments, the steroid comprises prednisone.

In embodiments, the effective amount of the umbilical lining-derived stem cells is at least about 50 million cells. In embodiments, the effective amount of the umbilical lining-derived stem cells is at least about 100 million cells. In embodiments, the effective amount of the umbilical lining-derived stem cells is at least about 200 million cells. In embodiments, the administering involves administering a single dose of the umbilical lining-derived stem cells. In other embodiments, the administering involves administering multiple doses of the umbilical lining-derived stem cells over a defined period of time.

In another aspect, a method of improving a symptom associated with idiopathic inflammatory myopathy in an individual is disclosed. The method involves administering an effective amount of umbilical lining-derived stem cells to the individual in need thereof.

In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of IMM (muscle weakness and autoantibodies), including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM).

In embodiments, the idiopathic inflammatory conditions may comprise polymyositis (PM), dermatomyositis (DM) (including juvenile, amyopathic, and sine-dermatitis form), inclusion-body myositis (IBM), immune-mediated necrotizing myopathy (IMNM), focal autoimmune myositis, Lambert-Eaton myasthenic syndrome, myasthenia gravis, the muscle form of sarcoidosis, idiopathic juvenile arthritis, idiopathic spondylarthrites, rheumatoid arthritis, psoriatic arthritis, LUPUS, idiopathic transverse myelitis, idiopathic optic neuritis, idiopathic intracranial hypertension, idiopathic pulmonary fibrosis (IPF), idiopathic interstitial pneumonias (umbrella group with several subtypes), cryptogenic organizing pneumonia (idiopathic form of bronchiolitis obliterans organizing pneumonia), idiopathic inflammatory bowel disease (IBD) including Crohn's disease, and ulcerative colitis, idiopathic granulomatous hepatitis, idiopathic urticaria (chronic spontaneous urticaria); idiopathic panniculitis; idiopathic vasculitis, idiopathic crescentic glomerulonephritis; idiopathic nephrotic syndrome, idiopathic pericarditis, idiopathic myocarditis, idiopathic aortitis, idiopathic systemic vasculitis's. In certain embodiments, idiopathic inflammation refers to any one or combination of conditions recited herein.

In another aspect a method is disclosed for identification of patients with inflammatory conditions that would otherwise remain undetected in a standard blood plasma assays such as C-Reactive Protein (CRP) and Erythrocyte Sedimentation rate (ESR).

In embodiments, the effective amount of the umbilical lining-derived stem cells is at least about 50 million cells. In embodiments, the effective amount of the umbilical lining-derived stem cells is at least about 100 million cells. In embodiments, the effective amount of the umbilical lining-derived stem cells is at least about 200 million cells.

In certain embodiments, the method further involves reducing or tapering a steroid dosage in the individual.

In embodiments, the steroid comprises prednisone, prednisolone, methyl prednisone, dexamethasone, betamethasone, or triamcinolone, or derivates thereof. In certain embodiments, the steroid comprises prednisone.

In embodiments, the administering involves administering a single dose of the umbilical lining-derived stem cells. In certain embodiments, the administering involves administering multiple doses of the umbilical lining-derived stem cells over a defined period of time.

In certain embodiments, the symptom improvement is measured over at least a 1-month duration. In certain embodiments, the symptom improvement is measured over at least a 2-month duration. In embodiments, the symptom improvement is measured over at least a 3-month duration.

In certain embodiments, the symptom improvement correlates with a decrease in a cytokine level in the individual. In certain embodiments, the symptom improvement correlates with a decrease in TNF-α levels in the individual. In certain embodiments, the symptom improvement correlates with a decrease in IL-12 levels in the individual. In certain embodiments, the symptom improvement correlates with a decrease in IL-12p40 levels in the individual. In certain embodiments, the symptom improvement correlates with a decrease in IL-12p70 levels in the individual. In certain embodiments, the symptom improvement correlates with a decrease in MIP-1α levels in the individual. In certain embodiments, the symptom improvement correlates with a decrease in IL-23 levels in the individual. In certain embodiments, the symptom improvement correlates with a decrease in IL-1β in the individual. In certain embodiments, the symptom improvement correlates with a decrease in Granzyme A in the individual. In certain embodiments, the symptom improvement correlates with a decrease in I-309 (CCL1) in the individual. In certain embodiments, the symptom improvement correlates with a decrease in MIP-1β (CCL4) in the individual. In certain embodiments, the symptom improvement correlates with a decrease in IL-8 in the individual. In certain embodiments, the symptom improvement correlates with a decrease in PDGF-AB/BB in the individual. In certain embodiments, the symptom improvement correlates with a decrease in IL-27 in the individual. In certain embodiments, the symptom improvement correlates with a decrease in IL-1α in the individual. In certain embodiments, the symptom improvement correlates with a decrease in IL-6 in the individual. In certain embodiments, the symptom improvement correlates with a decrease in EGF in the individual. In certain embodiments, the symptom improvement correlates with a decrease in IL-2 in the individual. In certain embodiments, the symptom improvement correlates with a decrease in IFNγ in the individual. In certain embodiments, the symptom improvement correlates with a decrease in TNFβ in the individual. In certain embodiments, the symptom improvement correlates with a decrease in MDC (CCL22) in the individual. In certain embodiments, the symptom improvement correlates with a decrease in IL-17A in the individual.

In another aspect, an effective amount of umbilical lining-derived stem cells for use in treating an individual diagnosed with idiopathic inflammation is disclosed.

In another aspect, an effective amount of umbilical lining-derived stem cells for use in treating an individual diagnosed with idiopathic inflammatory myopathy is disclosed.

In another aspect, an effective amount of umbilical lining-derived stem cells for use in treating an individual diagnosed with rheumatoid arthritis is disclosed.

In another aspect, an effective amount of umbilical lining-derived stem cells for use in treating an individual diagnosed with ulcerative colitis is disclosed.

In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of IMM (muscle weakness and autoantibodies), including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM).

In embodiments, the idiopathic inflammatory conditions may comprise polymyositis (PM), dermatomyositis (DM) (including juvenile, amyopathic, and sine-dermatitis form), inclusion-body myositis (IBM), immune-mediated necrotizing myopathy (IMNM), focal autoimmune myositis, Lambert-Eaton myasthenic syndrome, myasthenia gravis, the muscle form of sarcoidosis, idiopathic juvenile arthritis, idiopathic spondylarthrites, rheumatoid arthritis, psoriatic arthritis, LUPUS, idiopathic transverse myelitis, idiopathic optic neuritis, idiopathic intracranial hypertension, idiopathic pulmonary fibrosis (IPF), idiopathic interstitial pneumonias (umbrella group with several subtypes), cryptogenic organizing pneumonia (idiopathic form of bronchiolitis obliterans organizing pneumonia), idiopathic inflammatory bowel disease (IBD) including Crohn's disease, and ulcerative colitis, idiopathic granulomatous hepatitis, idiopathic urticaria (chronic spontaneous urticaria); idiopathic panniculitis; idiopathic vasculitis, idiopathic crescentic glomerulonephritis; idiopathic nephrotic syndrome, idiopathic pericarditis, idiopathic myocarditis, idiopathic aortitis, idiopathic systemic vasculitis's. In certain embodiments, idiopathic inflammation refers to any one or combination of conditions recited herein.

In certain embodiments, the effective amount comprises at least about 50 million cells. In certain embodiments, the effective amount comprises at least about 100 million cells. In certain embodiments, the effective amount comprises at least about 200 million cells.

All of these embodiments are intended to be within the scope of the invention herein disclosed. These and other embodiments will become readily apparent to those skilled in the art from the following detailed description of the preferred embodiments having reference to the attached figures, the invention not being limited to any particular preferred embodiment as disclosed.

### BRIEF DESCRIPTION OF THE DRAWINGS

FIG. 1 refers to a table containing certain Phase 1 trial results as described herein.

FIG. 2 refers to a graph showing Physician Global Disease Score data against defined periods of time.

FIG. 3 refers to a graph showing Physician Global Disease Score data against defined periods of time.

FIG. 4 refers to a graph showing Manual Muscle Testing (MMT-8) data against defined periods of time.

FIG. 5 refers to a graph showing Health Assessment Questionnaire Score data against defined periods of time.

FIG. 6 refers to a graph showing Average score data of Extramuscular Disease against defined periods of time.

FIG. 7 refers to a graph showing Total Improvement Score data against defined periods of time.

FIG. 8 refers to a graph showing Average Percent Reduction from Baseline Dose data against defined periods of time.

FIG. 9A refers to a graph depicting TNF-α (pf/mL) across various tested conditions for Patient 08.

FIG. 9B refers to a graph depicting TNF-α (pf/mL) across various tested conditions for Patient 09.

FIG. 10 refers to Average score data TIS (using a 1-10 scoring system) against defined periods of time. Average of all patients TIS which is a 1-10 score.

FIGS. 11A-11B refer to graphs illustrating significant elevations of IL-12 in patients with idiopathic inflammatory myopathy (IIM) as compared to normal patients without IIM.

FIG. 12 refers to a graph showing the average of IL-12p40 for IIM patients, showing that ULSCs reduce IL-12p40 in patients with IIM, and reduce the inflammatory response of patients' peripheral blood mononuclear cells (PBMCs) when stimulated with LPS.

FIG. 13 refers to a graph showing the average of IL-12p40 for IIM patients, showing that ULSCs reduce the IL-12p40 inflammatory response of patients' PBMCs when stimulated with inflammatory producing beads.

FIG. 14 refers to a graph showing the average of IL-12p70 for IIM patients, showing that ULSCs reduce the IL-12p70 inflammatory response of patients' PBMCs when stimulated with LPS.

FIG. 15 refers to a graph showing significant elevations of the cytokine macrophage inflammatory protein 1α (MIP-1α) in patients with IIM as compared to healthy controls.

FIG. 16 refers to a graph showing the average of MIP-1α for IIM patients, showing that ULSCs reduce the amount of MIP-1α in patients' PBMCs when stimulated with LPS.

FIG. 17 refers to a graph showing IL-23 levels for PBMC baseline, showing that patients with IIM have significantly higher levels of the cytokine IL-23 as compared to healthy controls.

FIG. 18 refers to a graph showing the average of IL-23 for IIM patients, showing that, in patients with IIM, PBMCs have significantly higher levels of IL-23, and when stimulated with LPS, IL-23 further increases significantly more.

FIG. 19 refers to a graph showing the average of IL-23 for IIM patients, showing that, in patients with IIM, PBMCs have significantly higher levels of IL-23, and when stimulated with immune stimulating beads, IL-23 further increases significantly more.

FIG. 20 refers to a graph showing baseline suppression of inflammatory factors in the supernatant of PBMCs after treatment with ULSCs.

FIG. 21 refers to a graph showing suppression of inflammatory factors in the supernatant of PBMCs antagonized by LPS and then treated with ULSCs.

FIG. 22 refers to a graph showing suppression of inflammatory factors in the supernatant of PBMCs antagonized by CD3 and CD28 beads and then treated with ULSCs.

FIG. 23 refers to a graph showing reduced inflammatory responses of white cells in clinical trials patients after treatment with ULSCs.

FIG. 24 refers to a graph showing suppression of inflammatory factors in the blood of an IIM patient after treatment with ULSCs.

FIG. 25 refers to a graph showing suppression of inflammatory factors in the blood plasma of a rheumatoid arthritis patient after treatment with ULSCs.

FIG. 26 refers to a graph showing suppression of inflammatory factors in the blood plasma of an ulcerative colitis patient after treatment with ULSCs.

### DETAILED DESCRIPTION

Although certain embodiments and examples are described below, those of skill in the art will appreciate that the invention extends beyond the specifically disclosed embodiments and/or uses and obvious modifications and equivalents thereof. Thus, it is intended that the scope of the invention disclosed herein should not be limited by any particular embodiments described below.

In an aspect, a method of treating idiopathic inflammatory myopathy in an individual is disclosed. The method involves administering an effective amount of umbilical lining-derived stem cells to the individual in need thereof. In embodiments, the administering involves administering a single dose of the umbilical lining-derived stem cells. In other embodiments, the administering involves administering multiple doses of the umbilical lining-derived stem cells over a defined period of time. The defined period of time may be two sequential days or may be spread out 1-, 2-, 3-, or 4-months apart. In certain embodiments, the defined period of time may exceed 4-months between doses.

In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of IMM (muscle weakness and autoantibodies), including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM).

In embodiments, the idiopathic inflammatory conditions may comprise polymyositis (PM), dermatomyositis (DM) (including juvenile, amyopathic, and sine-dermatitis form), inclusion-body myositis (IBM), immune-mediated necrotizing myopathy (IMNM), focal autoimmune myositis, Lambert-Eaton myasthenic syndrome, myasthenia gravis, the muscle form of sarcoidosis, idiopathic juvenile arthritis, idiopathic spondylarthrites, rheumatoid arthritis, psoriatic arthritis, LUPUS, idiopathic transverse myelitis, idiopathic optic neuritis, idiopathic intracranial hypertension, idiopathic pulmonary fibrosis (IPF), idiopathic interstitial pneumonias (umbrella group with several subtypes), cryptogenic organizing pneumonia (idiopathic form of bronchiolitis obliterans organizing pneumonia), idiopathic inflammatory bowel disease (IBD) including Crohn's disease, and ulcerative colitis, idiopathic granulomatous hepatitis, idiopathic urticaria (chronic spontaneous urticaria); idiopathic panniculitis; idiopathic vasculitis, idiopathic crescentic glomerulonephritis; idiopathic nephrotic syndrome, idiopathic pericarditis, idiopathic myocarditis, idiopathic aortitis, idiopathic systemic vasculitis's. In certain embodiments, idiopathic inflammation refers to any one or combination of conditions recited herein.

In embodiments, the effective amount of the umbilical lining-derived stem cells is less than about 50 million cells. For example, the effective amount may be 45 million cells, 40 million cells, 35 million cells, 30 million cells, 25 million cells, 20 million cells, 15 million cells, 10 million cells, or fewer than 10 million cells. In certain embodiments, the effective amount of the umbilical lining-derived stem cells is at least about 50 million cells. In embodiments, the effective amount is at least 60 million cells, at least 70 million cells, at least 80 million cells, or at least 90 million cells. In embodiments, the effective amount of the umbilical lining-derived stem cells is at least about 100 million cells. In embodiments, the effective amount is at least 110 million cells, at least 120 million cells, at least 130 million cells, at least 140 million cells, at least 150 million cells, at least 160 million cells, at least 170 million cells, at least 180 million cells, or at least 190 million cells. In embodiments, the effective amount of the umbilical lining-derived stem cells is at least about 200 million cells. In embodiments, the effective amount is in excess of 200 million cells.

In another aspect, a method of reducing steroid usage in an individual afflicted with idiopathic inflammatory myopathy is disclosed. The method involves administering an effective amount of umbilical lining-derived stem cells to the individual in need thereof. In embodiments, the administering involves administering a single dose of the umbilical lining-derived stem cells. In other embodiments, the administering involves administering multiple doses of the umbilical lining-derived stem cells. In some embodiments, the administering involves administering multiple doses of the umbilical lining-derived stem cells over a defined period of time.

In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of IMM (muscle weakness and autoantibodies), including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM). In embodiments, the steroid comprises prednisone, prednisolone, methyl prednisone, dexamethasone, betamethasone, or triamcinolone, or derivates thereof. In certain embodiments, the steroid comprises prednisone. In embodiments, the effective amount of the umbilical lining-derived stem cells is less than about 50 million cells. For example, the effective amount may be 45 million cells, 40 million cells, 35 million cells, 30 million cells, 25 million cells, 20 million cells, 15 million cells, 10 million cells, or fewer than 10 million cells. In embodiments, the effective amount of the umbilical lining-derived stem cells is at least about 50 million cells. In embodiments, the effective amount is at least 60 million cells, at least 70 million cells, at least 80 million cells, or at least 90 million cells. In embodiments, the effective amount of the umbilical lining-derived stem cells is at least about 100 million cells. In embodiments, the effective amount is at least 110 million cells, at least 120 million cells, at least 130 million cells, at least 140 million cells, at least 150 million cells, at least 160 million cells, at least 170 million cells, at least 180 million cells, or at least 190 million cells. In embodiments, the effective amount of the umbilical lining-derived stem cells is at least about 200 million cells. In embodiments, the effective amount is in excess of 200 million cells.

In another aspect, a method of tapering steroid usage in an individual afflicted with idiopathic inflammatory myopathy is disclosed. The method involves administering an effective amount of umbilical lining-derived stem cells to the individual in need thereof. In embodiments, the administering involves administering a single dose of the umbilical lining-derived stem cells. In other embodiments, the administering involves administering multiple doses of the umbilical lining-derived stem cells. In some embodiments, the administering involves administering multiple doses of the umbilical lining-derived stem cells over a defined period of time.

In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of IMM (muscle weakness and autoantibodies), including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM). In embodiments, the steroid comprises prednisone, prednisolone, methyl prednisone, dexamethasone, betamethasone, or triamcinolone, or derivates thereof. In certain embodiments, the steroid comprises prednisone.

In embodiments, the effective amount of the umbilical lining-derived stem cells is less than about 50 million cells. For example, the effective amount may be 45 million cells, 40 million cells, 35 million cells, 30 million cells, 25 million cells, 20 million cells, 15 million cells, 10 million cells, or fewer than 10 million cells. In embodiments, the effective amount of the umbilical lining-derived stem cells is at least about 50 million cells. In embodiments, the effective amount is at least 60 million cells, at least 70 million cells, at least 80 million cells, or at least 90 million cells. In embodiments, the effective amount of the umbilical lining-derived stem cells is at least about 100 million cells. In embodiments, the effective amount is at least 110 million cells, at least 120 million cells, at least 130 million cells, at least 140 million cells, at least 150 million cells, at least 160 million cells, at least 170 million cells, at least 180 million cells, or at least 190 million cells. In embodiments, the effective amount of the umbilical lining-derived stem cells is at least about 200 million cells. In embodiments, the effective amount is in excess of 200 million cells.

In another aspect, a method of improving a symptom associated with idiopathic inflammatory myopathy in an individual is disclosed. The method involves administering an effective amount of umbilical lining-derived stem cells to the individual in need thereof.

In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of IMM (muscle weakness and autoantibodies), including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM). In embodiments, the effective amount of the umbilical lining-derived stem cells is at least about 50 million cells. In embodiments, the effective amount of the umbilical lining-derived stem cells is at least about 100 million cells. In embodiments, the effective amount of the umbilical lining-derived stem cells is at least about 200 million cells. In certain embodiments, the method further involves reducing a steroid dosage in the individual. In embodiments, the steroid comprises prednisone, prednisolone, methyl prednisone, dexamethasone, betamethasone, or triamcinolone, or derivates thereof. In certain embodiments, the steroid comprises prednisone. In embodiments, the administering involves administering a single dose of the umbilical lining-derived stem cells. In other embodiments, the administering involves administering multiple doses of the umbilical lining-derived stem cells. In some embodiments, the administering involves administering multiple doses of the umbilical lining-derived stem cells over a defined period of time. In certain embodiments, the symptom improvement is measured over at least a 1-month duration. In certain embodiments, the symptom improvement is measured over at least a 2-month duration. In embodiments, the symptom improvement is measured over at least a 3-month duration. In embodiments, the symptom improvement is measured over a period extending beyond 3-months. In certain embodiments, the symptom improvement correlates with a decrease in TNF-α levels in the individual. In certain embodiments, the symptom improvement correlates with a decrease in a cytokine level in the individual. In certain embodiments, the cytokine comprises IL-12. In certain embodiments, the cytokine comprises IL-12p40. In certain embodiments, the cytokine comprises IL-12p70. In certain embodiments, the cytokine comprises MIP-1α. In certain embodiments, the cytokine comprises IL-23.

In another aspect, an effective amount of umbilical lining-derived stem cells for use in treating an individual diagnosed with idiopathic inflammatory myopathy is disclosed. In embodiments, the idiopathic inflammatory myopathy comprises polymyositis (PM), dermatomyositis (DM) (including juvenile, amyopathic, and sine-dermatitis form), inclusion-body myositis (IBM), immune-mediated necrotizing myopathy (IMNM), and focal autoimmune myositis. Other autoimmune diseases can mimic the symptoms of IMM (muscle weakness and autoantibodies), including Lambert-Eaton myasthenic syndrome, myasthenia gravis, and the muscle form of sarcoidosis, and myositis in general. In certain embodiments, the idiopathic inflammatory myopathy comprises polymyositis (PM). In certain embodiments, the idiopathic inflammatory myopathy comprises dermatomyositis (DM). In embodiments, the effective amount of the umbilical lining-derived stem cells is less than about 50 million cells. For example, the effective amount may be 45 million cells, 40 million cells, 35 million cells, 30 million cells, 25 million cells, 20 million cells, 15 million cells, 10 million cells, or fewer than 10 million cells. In certain embodiments, the effective amount of the umbilical lining-derived stem cells is at least about 50 million cells. In embodiments, the effective amount is at least 60 million cells, at least 70 million cells, at least 80 million cells, or at least 90 million cells. In embodiments, the effective amount of the umbilical lining-derived stem cells is at least about 100 million cells. In embodiments, the effective amount is at least 110 million cells, at least 120 million cells, at least 130 million cells, at least 140 million cells, at least 150 million cells, at least 160 million cells, at least 170 million cells, at least 180 million cells, or at least 190 million cells. In embodiments, the effective amount of the umbilical lining-derived stem cells is at least about 200 million cells. In embodiments, the effective amount is in excess of 200 million cells.

Mesenchymal stem/stromal cells (MSCs) are self-renewing, multi-potent stromal cells which act as modulators of immune responses: they have been shown to inhibit dendritic cell (DC) maturation (Spaggiari, G. M., et al., Blood, 2009. 113(26): p. 6576-83) and their ability to present antigen to T cells (Chiesa, S., et al., Proc Natl Acad Sci USA, 2011. 108(42): p. 17384-9); to block B-cell differentiation into plasma cells (Franquesa, M., et al., Stem Cells, 2015. 33 (3): p. 880-91) and antibody production (Asari, S., et al., Exp Hematol, 2009. 37(5): p. 604-15.); as well as to reduce Th17 and Th1 in favor of T regulatory cell responses (Ghannam, S., et al., The Journal of Immunology, 2010. 185(1): p. 302-312).

These MSC activities do not generally require cell-cell contact, and are mediated by multiple secreted factors, including PGE2 (Spaggiari et al., 2009), TGF-β (English, K., et al., Clin Exp Immunol, 2009. 156(1): p. 149-60) and IL-10 (Lee, R. H., et al., Proc Natl Acad Sci U SA, 2014. 111(47): p. 16766-71). As a consequence of these pleiotropic activities, MSCs have been shown to possess potent anti-inflammatory effects in animal models of auto-immune disease, including models of colitis (Gonzalez, M. A., et al., Gastroenterology, 2009. 136(3): p. 978-89), graft-versus-host disease (Yanez, R., et al., Stem Cells, 2006. 24(11): p. 2582-91), and rheumatoid arthritis (Gonzalez-Rey, E., et al., Ann Rheum Dis, 2010. 69(1): p. 241-8). In addition, MSCs are poorly immunogenic, as they lack costimulatory CD80 and poorly express human leukocyte antigen (HLA) class II surface antigens (See, F., et al., J Cell Mol Med, 2011. 15(10): p. 2117-29).

MSCs can be obtained through bone marrow aspiration, liposuction, or from the umbilical cord. The last, termed umbilical cord-derived mesenchymal stromal cells (U-MSC), present multiple advantages: they are obtained from a medical waste material, which does not raise ethical concerns; their origin may be associated with a younger cellular phenotype; and studies indicate minimal expression of HLA-DR after activation, suggesting they have a reduced immunogenic potential (Kim, J. H., et al., Stem Cells Int, 2018. 2018: p. 8429042).

In the practice of the embodiments herein, human umbilical cord stem cells are used as a source of ULSCs. Umbilical mesenchymal stem cells suitable for use in the methods and compositions described here may come from a variety of sources. Umbilical mesenchymal stem cells are reviewed in: Troyer et al., Concise Review: Wharton's Jelly-Derived Cells are a Primitive Stromal Cell Population, Stem Cells, 2008, 26:591-599). Any of the umbilical mesenchymal stem cells described in that document may be employed in the methods and compositions described here.

In some embodiments the UC MSCs used are umbilical cord lining stem cells (ULSCs) positive for expression of the cell surface markers CD105, CD106, CD90, CD73, SSEA-4, and STRO-1, negative for CD45, CD34, CD19, and HLA-DR, express OCT4 and Nanog, and do not express Sox2. In another aspect of the embodiment the ULSCs used are multipotent capable of differentiation into cells of mesodermal lineage (e.g., adipogenic cells, osteogenic cells, chondrogenic, and cardiogenic cells) or ectodermal lineage (e.g., neurogenic cells). In a further aspect of the embodiment, the ULSCs may undergo at least 50, 60, 70, 80 or 90 doublings in culture.

In one embodiment, patient blood is evaluated for inflammatory factors in vitro by the following procedures: Required reagents and equipment: ULSCs (culture-expanded, passage 3-4), fresh human PBMCs from donors with known inflammatory conditions (isolated via Ficoll gradient), RPMI+5% FBS (PBMC medium), ULSCs growth medium (e.g., DMEM/F12+5% FBS), Trypsin/EDTA, LPS (for monocyte activation), CD3/CD28 activation beads (for T-cell activation), 24-well plates, Standard tissue culture equipment. Step 1. ULSCs Preparation: Thaw ULSCs (P3-4) and plate at ˜3,000 cells/cm² in ULSCs medium 48 hours before assay. On assay day, detach with trypsin, neutralize, count, and plate ˜35,000 ULSCs/well in a 24-well plate (0.7 mL per well). Incubate 3 hours before adding PBMCs. Step 2. PBMC Isolation: Collect fresh human blood in EDTA tubes. Dilute with PBS and layer over Ficoll; centrifuge at 400 g for 30 minutes. Harvest PBMC layer, wash twice in PBS, and resuspend in RPMI+5% FBS. Adjust to ˜350,000 live PBMCs/well. Step 3. Stimulation Conditions: Prepare PBMC suspensions for: Unstimulated (PBMC+media), LPS-stimulated (10 ng/ml final), CD3/CD28 bead-stimulated (1 bead: 2 PBMCs), Step 4. Co-Culture Setup: Remove ULSCs culture medium, Add 0.7 mL of each PBMC suspension to ULSCs wells (triplicates). Set up parallel PBMC-only wells as controls. Incubate 24 hours at 37° C., 5% CO₂. Step 5. Harvest & Analysis: Collect supernatants, centrifuge at 300 g×7 minutes, transfer to clean tubes. Aliquot and freeze at −80° C. Analyze cytokines (e.g., TNF-α, IL-12, IL-12p40, IL-12p70, MIP-1α, IL-23, IL-1β, Granzyme A, I-309 (CCL1), MIP-1β (CCL4), IL-8, PDGF-AB/BB, IL-27, IL-1α, IL-6, EGF, IL-2, IFNγ, TNFβ, MDC (CCL22), IL-17A.) via ELISA or multiplex assay.

Non-randomized, single arm clinical trials employing intravenous administration of MSCs derived from bone-marrow or umbilical cord have demonstrated an excellent safety profile for cellular therapy in drug-resistant systemic lupus erythematosus (81 patients followed for ≥5 years) (Wang, D., et al., Stem Cell Reports, 2018. 10(3): p. 933-941), drug-resistant dermatomyositis or polymyositis (15 patients followed for ≥12 months) (Wang, D., et al., Ann Rheum Dis, 2011. 70(7): p. 1285-8) and in idiopathic pulmonary fibrosis (8 patients followed for 6 months) (Chambers, D. C., et al., Respirology, 2014. 19(7): p. 1013-8). Although most of these studies reported signs of therapeutic efficacy, placebo-controlled studies are required to tease out the benefit of MSC therapy from that of standard, optimized medical therapy.

DM and PM have a complex pathophysiology that may be efficiently counteracted by MSC therapy at various levels. In affected muscles, micro-vascular injury is the first pathological change, preceding perimysial muscle fiber atrophy (Hornung, T. and J. Wenzel, Drugs, 2014. 74(9): p. 981-98). Histological analysis of biopsy samples from affected muscles in DM and PM patients show evidence of endothelial cell sloughing and consequent deposition of membrane attack complexes (Hornung et al., 2014; Lahoria, R., D. Selcen, and A. G. Engel, Brain, 2016. 139(Pt 7): p. 1891-903). MSCs may effectively reduce this microvascular injury pattern, as they have been shown to rescue endothelial cell function in multiple disease scenarios (Feng, Y., et al., 2019. 28(10): p. 674-682; Premer, C., et al., EBioMedicine, 2015. 2(5): p. 467-75); they also promote angiogenesis by producing growth factors such as VEGF (Feng et al., 2019), HGF (Kwon, H. M., et al., Vascul Pharmacol, 2014. 63(1): p. 19-28), or MCP-1 (Kwon et al., 2014), which may counteract the microvascular rarefaction seen in DM/PM. An abnormally up-regulated type-I interferon (IFN) signature is a hallmark of DM and PM (Hornung et al., 2014); plasmacytoid DCs (pDCs) are an important source of type-I interferons (Wenzel, J., et al., Clin Exp Dermatol, 2006. 31(4): p. 576-82), and infiltrating pDCs are abundant in biopsies from diseased muscle (Greenberg, S. A., et al., Ann Neurol, 2005. 57(5): p. 664-78). MSCs may reduce pDC type I IFN production in DM/PM by virtue of their capacity to inhibit DC differentiation, maturation and activation (Spaggiari et al., 2009; Chiesa et al., 2011). MSCs may also reduce the production of autoantibodies, which act as stimulatory factors for pDC IFN production. In this regard, MSC have been shown to inhibit B cell maturation into plasmacytoid (Spaggiari et al., 2009) cells, as well as auto-antibody production. Additionally, they may reduce auto-antibody production by enhancing T regulatory cells development (English et al., 2009; Carrion, F., et al., Lupus, 2010. 19(3): p. 317-22; Luz-Crawford, P., et al., Mesenchymal stem cells generate a CD4+CD25+Foxp3+ regulatory T cell population during the differentiation process of Th1 and Th17 cells. Stem Cell Res Ther, 2013. 4(3): p. 65), which block the activation of allo-reactive Th1 cells (Carneiro, J., et al., Immunol Rev, 2007. 216: p. 48-68; Leon, K., et al., Modelling T-cell-mediated suppression dependent on interactions in multicellular conjugates. J Theor Biol, 2000. 207(2): p. 231-54). Other dysfunctional T cell responses noted in DM/PM may also be effectively modulated by MSC. CD28 cells are increased in the peripheral blood of DM/PM patients, represent the majority of infiltrating CD4+ and CD8+ T cells infiltrating muscle (Fasth, A. E., et al., J Immunol, 2009. 183(7): p. 4792-9), and persist in muscle after glucocorticoid treatment (Pandya, J. M., et al., Arthritis Res Ther, 2016. 18: p. 80). They are resistant to apoptosis, and have augmented cytotoxicity and cytokine-production capabilities (Venalis, P. and I. E. Lundberg, Rheumatology (Oxford), 2014. 53(3): p. 397-405). MSCs have been shown to block allo-reactive CD8+CD28 T cells obtained from human recipients of kidney transplant IL-17 production, which is mainly orchestrated by Th17 cells, is increased in DM/PM affected muscles, and this may be efficiently reduced by MSC-induced downregulation of Th17 cells (Venalis et al., 2014).

### Total Improvement Score (TIS)

Prior to initial enrollment at the beginning of the trial a meeting is held by an appropriate trainer that understands patient assessments, including the MDAAT and other tools described below, in order to optimize homogeneity of assessments throughout the protocol.

When assessing patient scores for physician-led tools, physicians are instructed to consider the prior results as anchors to ensure greater longitudinal consistency. Additionally, the same physician should, as much as feasible, fill out the same assessments at each site. All endpoint-based assessment timepoints are required to be completed by the same investigator who completed the baseline assessment; if this cannot be done it will be considered a protocol deviation. Secondary efficacy-related endpoints include the absolute value as well as the change from baseline at each post-treatment study visit listed in the Schedule in:
Core set measures that comprise the 2016 ACR/EULAR Myositis Response Criteria for Minimal, Moderate, and Major Clinical Response in Adult Dermatomyositis and Polymyositis:
   Physician global disease activity (VAS)
   Patient global disease activity (VAS)
   Manual muscle strength test (MMT)
   Health assessment questionnaire disability index (HAQ-DI)
   Extramuscular disease activity (VAS)
Myositis Disease Activity Assessment Tool (MDAAT)
Steroids
3D-TTE with speckle tracking and strain if available
Spirometry
FI-3

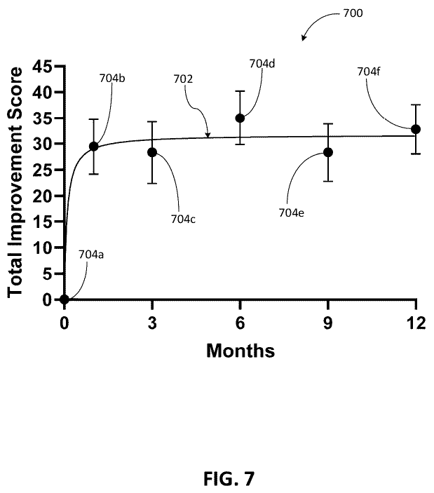

The TIS is not a separate assessment from the previously described Core Set Measures, but is automatically calculated as a composite of these measures, with the addition of muscle enzyme labs, using a web-based tool (see FIG. 7 herein). The results are computed as a change between timepoints where the improvement compared to a given initial timepoint is described as a percentage, with a conventional agreement that a minimally significant TIS is ≥20%. Using this approach, 100% represents complete improvement and 0% would represent no improvement, as a continuous variable.

We will assess disease activity using the Total Improvement Score (TIS) developed by the International Myositis Assessment & Clinical Studies Group, the American College of Rheumatology and the European League against rheumatism. The primary efficacy end-point will measure the magnitude of change in TIS before IV infusion of U-MSC and 6 months after, within each dosing group. The TIS is calculated on a scale from 0 to 100, using the “The Final 2016 ACR/EULAR Criteria for Minimal, Moderate, and Major Clinical Response in Adult Dermatomyositis and Polymyositis” as shown in Table 1:

TABLE 1
The Final 2016 ACR/EULAR Criteria for Minimal, Moderate, and Major
Clinical Response in Adult Dermatomyositis and Polymyositis

| | | Improvement |
|---|---|---|
| | Level of absolute % change in core set | score for |
| Core set measures | measures (improvement) | each level |
| Physician Global | Worsening to ≤5% improvement | 0 |
| Disease Activity | >5% up to ≤15% | 7.5 |
| | >15% up to ≤25% | 15 |
| | >25% up to ≤40% | 17.5 |
| | >40% | 20 |
| Patient Global | Worsening to ≤5% improvement | 0 |
| Disease Activity | >5% up to ≤15% | 2.5 |
| | >15% up to ≤25% | 5 |
| | >25% up to ≤40% | 7.5 |
| | >40% | 10 |
| Manual muscle | Worsening or no change (-ve change | 0 |
| strength test | to ≤5%) | |
| (MMT) | >5% up to ≤15% | 10 |
| | >15% up to ≤25% | 20 |
| | >25% up to ≤40% | 27.5 |
| | >40% | 32.5 |
| Health assessment | Worsening to ≤5% improvement | 0 |
| questionnaire | >5% up to ≤15% | 5 |
| disability index | >15% up to ≤25% | 7.5 |
| | >25% up to ≤40% | 7.5 |
| | >40% | 10 |
| Muscle enzyme | Worsening to ≤5% improvement | 0 |
| (most | >5% up to ≤15% | 2.5 |
| abnormal) | >15% up to ≤25% | 5 |
| | >25% up to <40% | 7.5 |
| | >40% | 7.5 |
| Extramuscular | Worsening to ≤5% improvement | 0 |
| disease activity | >5% up to ≤15% | 7.5 |
| | >15% up to ≤25% | 12.5 |
| | >25% up to ≤40% | 15 |
| | >40% | 20 |

Total Improvement Score (Scale 0-100)

### Physician Global Activity VAS Results

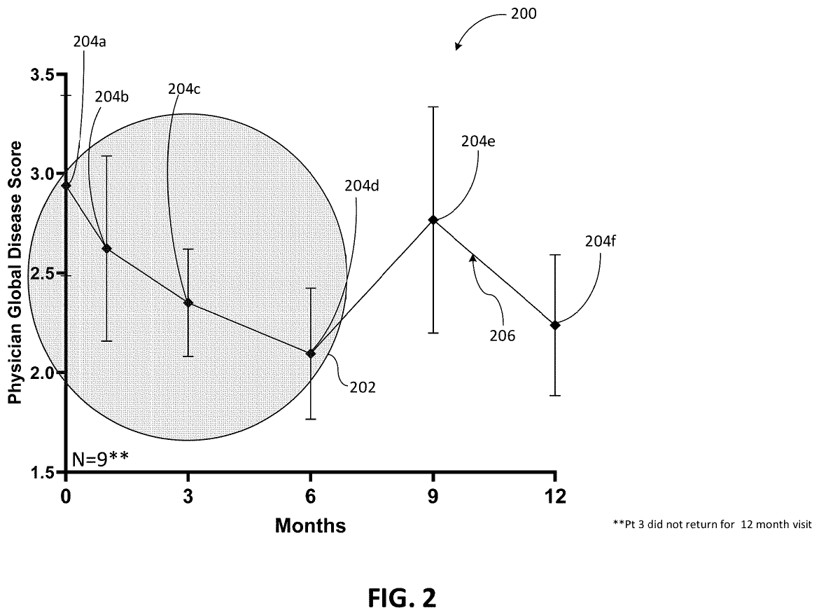

FIG. 2 provides longitudinal results of the Physician Global Activity (PGA) VAS scale for all 9 participants, regardless of dose level. The results suggest that symptomatic pain relief is identified in all dose cohorts through Month 6. The average baseline PGA score across all participants was 2.94 (+) 0.45 (mean (+) SEM), which by Month 6 had changed to 2.09 (+) 0.33. The PGA VAS scale extends from 0.0-10.0 cm, and is generally considered to provide clinically meaningful change with an improvement of ≥20% [45].

### MMT8 Scores

Manual Muscle Testing (MMT8) is a score evaluating the strength of a set of 8 designated muscles tested unilaterally, with a score range of 0-80. In this Phase 1 IIM study, participants demonstrated an overall improvement between baseline and Month 6 of nearly 10 points on the scale, with no SEM overlap: the values were 59.33±3.92 at baseline and 68.11±3.85 at Month 6 (mean±SEM).

The HAQ Disability Index (HAQ-DI) indicates the extent of the respondent's functional ability and is scored with the overall average range between 0 (no disability) to 3 (completely disabled). In the Phase 1 IIM study, participants demonstrated an overall change between baseline and Month 6 of −0.4 on the scale: these were 1.7 at baseline and 1.3 at Month 6. The Minimal Clinical Important Difference (MCID) for this scale has been variably reported as between 0.22 and 0.10. Accordingly, the average change from baseline in this trial was clinically significant throughout the first 6 months.

The TIS is a composite outcome derived from the six core set measures. The results are computed as a change between timepoints where the improvement compared to a given initial timepoint is described as a percentage, with a conventional agreement that a minimally significant TIS is ≥20%. Between baseline and one month following infusion of ULSC, 6 of the 9 participants manifested clinically significant improvement in disease based on the TIS score. Notably, these improvements were observed in the context of reduced prednisone dosage described above.

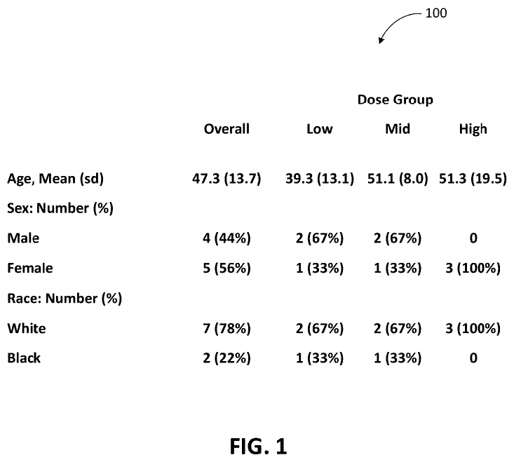

FIG. 1 at 100 provides a summary description of patients with PM/DM involved in the data capture. The average age of all participants in the study was 47.3 years of age with 56% being female and 44% being male. Additionally, 78% of participants were white, and 22% of participants were black. Three cohorts of participants are identified who received a low, mid, or high dose of ULSCs containing 50, 100, and 200 million cells respectively.

FIG. 2 at 200 provides an illustration of the Physician Global Disease Score in participants who were administered ULSCs. Elements 204a through 204d demonstrate an improvement from baseline to 6 months in participants with PM/DM. The Physician Global Disease Score is commonly used in autoimmune diseases that due to their prevalence in the Visual Analog Scale which is psychometric response scale that rates disease-related symptom severity on a scale of 1-10, with 10 being the worst. The assessment is conducted by a physician based on 42 scenarios based on patient synopsis. For example, patients are asked “Considering all the ways your disease has affected you in the last week, how active do you feel your disease is?”; or “Considering all the ways your rheumatic disease/myositis has affected you, how do you feel your rheumatic disease/myositis is today?” or in the “past week?”; or “How have you been feeling in general this past week, in relation to your rheumatic disease/myositis?”. Physician Global Activity (PGA) was measured on a visual analogue scale (VAS) ranging from 0 to 100 mm where higher scores represent worse subjective health. Elements 204a through 204f illustrate the varying score over a 12-month period.

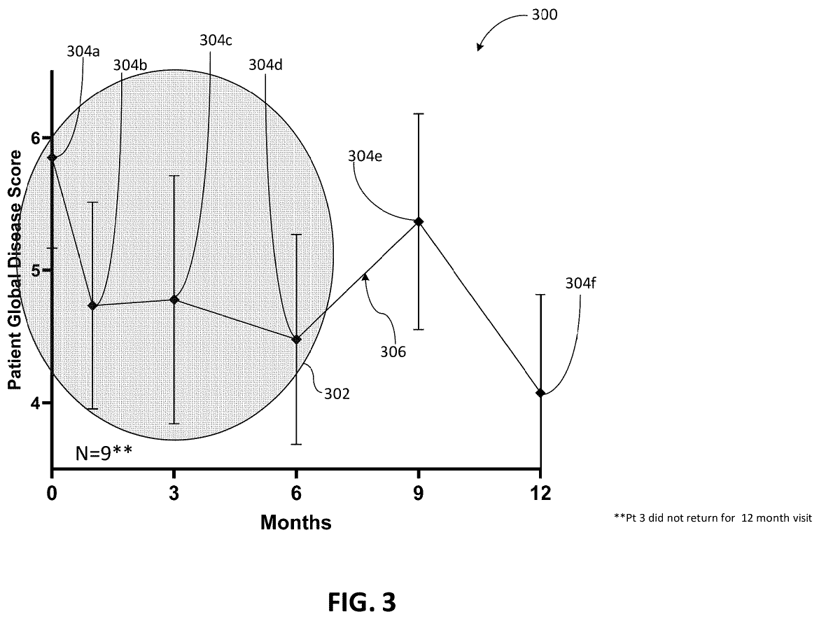

FIG. 3 at 300 provides an illustration the Patient Global Disease Score in participants with PM/DM who were administered ULSCs. Similar to the Physician Global Disease Score, there is a noted improvement from baseline to 6 months following administration. The Patient Global Disease Score is a patient reported outcome commonly used in autoimmune diseases. It is scored from 1-10, with 10 being the worse disease activity. The scoring is based on a patient assessment of themselves based on 42 scenarios. Elements 304a through 304f illustrate the varying score over a 12-month period.

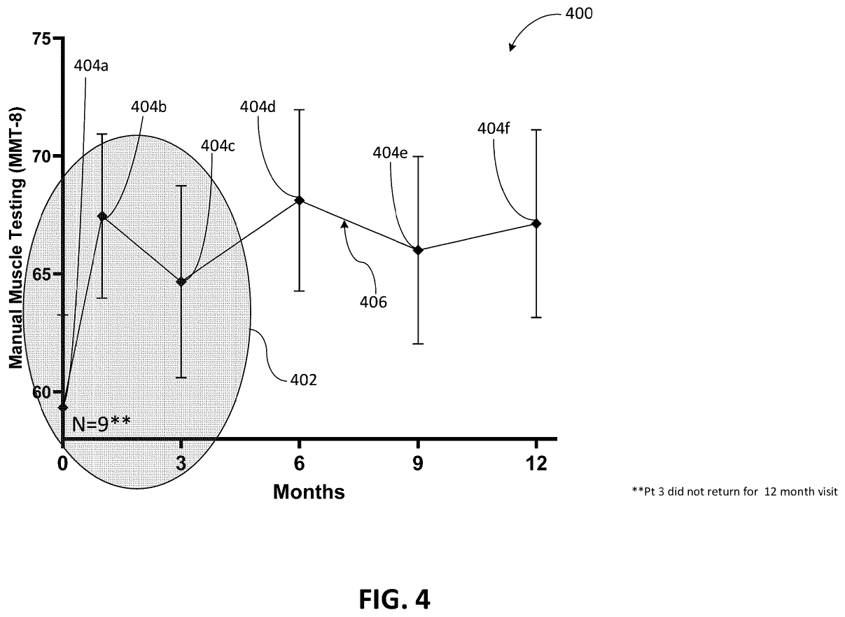

FIG. 4 at 400 provides an illustration with the average manual muscle testing (MMT-8) improvement for all participants who were administered ULSCs and maintained treatment from 1 month to 12 months. The improvement is compared to the baseline score prior to administration of ULSCs. The manual muscle testing (MMT-8) measure strength by the manual testing of 8 muscle groups. Strength is ranked from 0-80 with 80 being the highest muscle strength score. Elements 404a through 404f illustrate the varying score over a 12-month period.

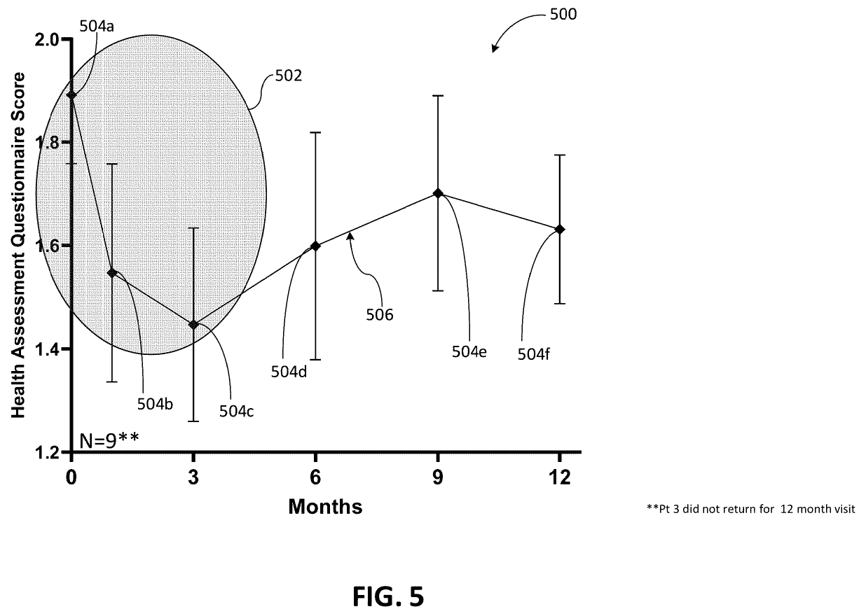

FIG. 5 at 500 provides an illustration of patient health following administration of ULSCs using the Health Assessment Questionnaire Disease Index (HAG-DI). The HAQ-DI is a patient reported outcome score that is ranked from 0-3, where 3 means completely disable and 0 means no disability. Elements 504a-504c exemplify a significant improvement in the HAQ from baseline to 3 months post-administration of ULSCs. Elements 504a through 504f display a general lowering of the HAQ-DI over 12 months as compared to baseline.

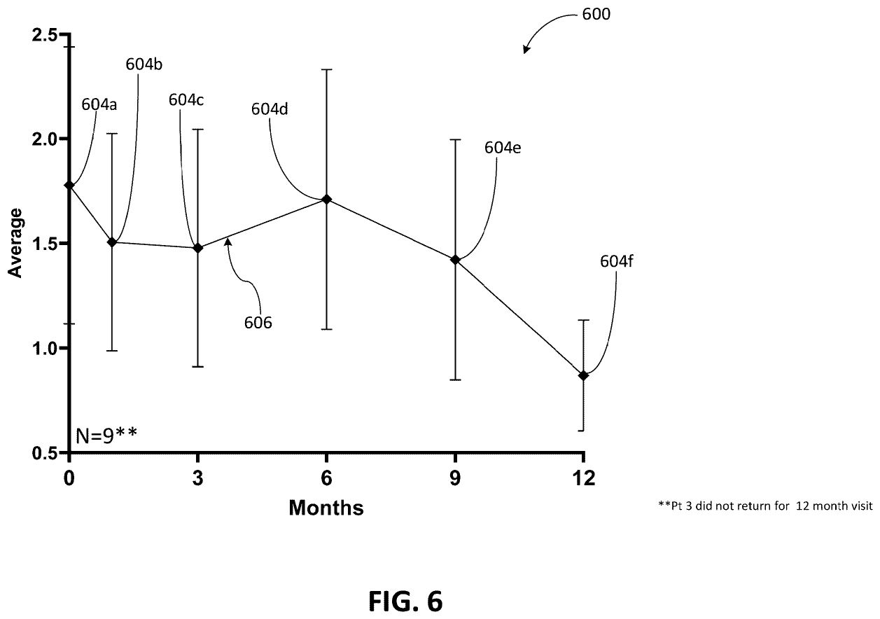

Referring to FIG. 6 at 600 provides an illustration representing the assessment of the average extra-muscular disease activity in all participants. The scoring system is ranked from 1-10 with higher scores representing more extra-muscular disease activity based on clinically defined assessments. Elements 604a-604f shows a significant lowering in extra-muscular disease activity score from baseline to 12 months post-administration of ULSCs which indicates less muscle disease. Elements 604a through 604f illustrate the varying score over a 12-month period.

FIG. 7 at 700 provides a graph displaying the total improvement score (TIS) of all participants over the course of a year. Elements 704a-704b show a significant increase one month after administration of ULSCs compared to the baseline score. This increase is maintained for the full length of the study. The TIS consists of a composite outcome from six core set measurements where the results are computed as a change between timepoints—where the improvement compared to a given initial timepoint is described as a percentage. The conventional agreement is that a minimally significant TIS is ≥20%. Between baseline and one-month post-administration of ULSCs, 6 of the 9 participants manifested clinically significant improvement in disease based on the TIS score. These improvements were observed in the context of reduced prednisone dosage. Elements 704a through 704f illustrate the varying score over a 12-month period.

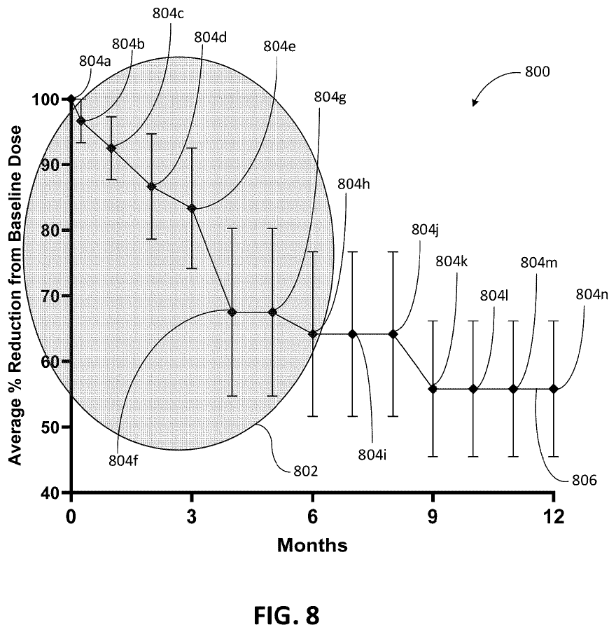

FIG. 8 at 800 provides a graph illustrating the average percent in reduction of prednisone between the baseline dose and up to 12 months post-administration of ULSCs. The average daily dose of prednisone was able to be tapered by more than 5.5 mg and the chronic use of as little as 2.6 mg daily lead to increased adverse side effects. Elements 804a through 804n illustrate the varying percentage reduction over a 12-month period.

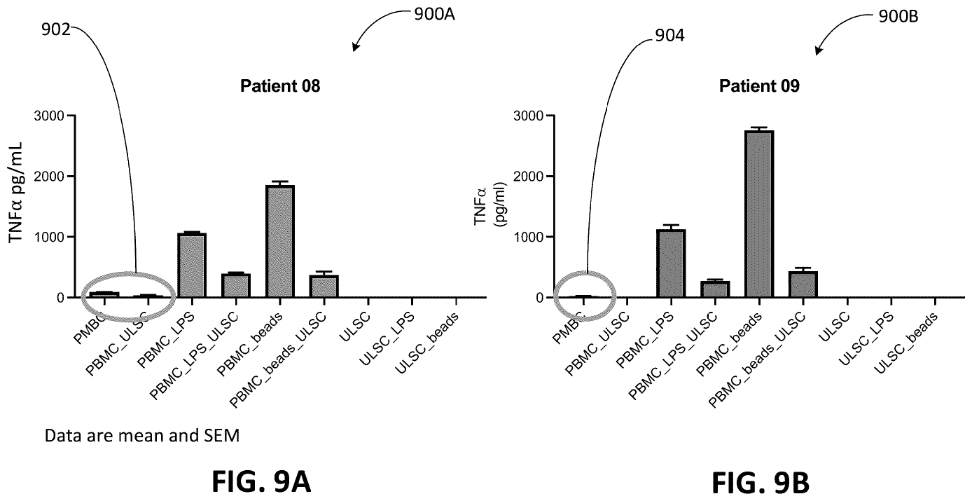

FIG. 9A at 900A provides an illustration of an in vitro assay mixing peripheral blood mononuclear cells (PBMC) from patient 08 with PM/DM with ULSCs to assess TNF-α. Before cell therapy, patients had a higher baseline release of TNF-α, but stimulated TNF-α release was repressed when PMBC was mixed with ULSCs. This pattern sustains even in groups where PBMC are stimulated with either lipopolysaccharide (LPS) or beads to secrete more TNF-α. TNF-α were assessed in conjunction with PBMC, PBMC plus ULSCs, PBMC stimulated with LPS, PBMC stimulated with beads and ULSCs, ULSCs alone, ULSCs stimulated with LPS, and ULSCs stimulated with beads.

FIG. 9B at 900B provides an illustration of an in vitro assay mixing peripheral blood mononuclear cells (PBMC) from patient 09 with PM/DM with ULSCs to assess TNF-α. Before cell therapy, patients had a higher baseline release of TNF-α, but stimulated TNF-α release was repressed when PMBC was mixed with ULSCs. This pattern sustains even in groups where PBMC are stimulated with either LPS or beads to secrete more TNF-α. TNF-α were assessed in conjunction with PBMC, PBMC plus ULSCs, PBMC stimulated with LPS, PBMC stimulated with beads and ULSCs, ULSCs alone, ULSCs stimulated with LPS, and ULSCs stimulated with beads.

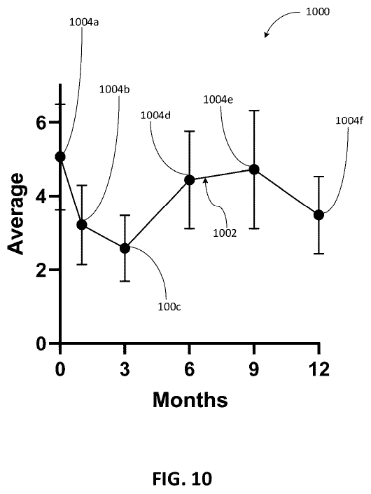

FIG. 10 at 1000 provides a graph illustrating the average reduction in pain for PM/DM participants from baseline scores to 12 months following administration of ULSCs. The pain scale is a patient reported outcome scale from 0-10 where 10 is the most pain and 0 is no reported pain. Elements 1004a through 1004f exemplify a general reduction in the amount of pain in PM/DM patients following administration of ULSCs. Elements 1004a through 1004c show that pain is reduced between 0 and 3 months before trending back up to baseline at 6 months.

ULSCs Regulation of Specific Analytes in Patients with IIM IL-12 Levels in IIM Patients

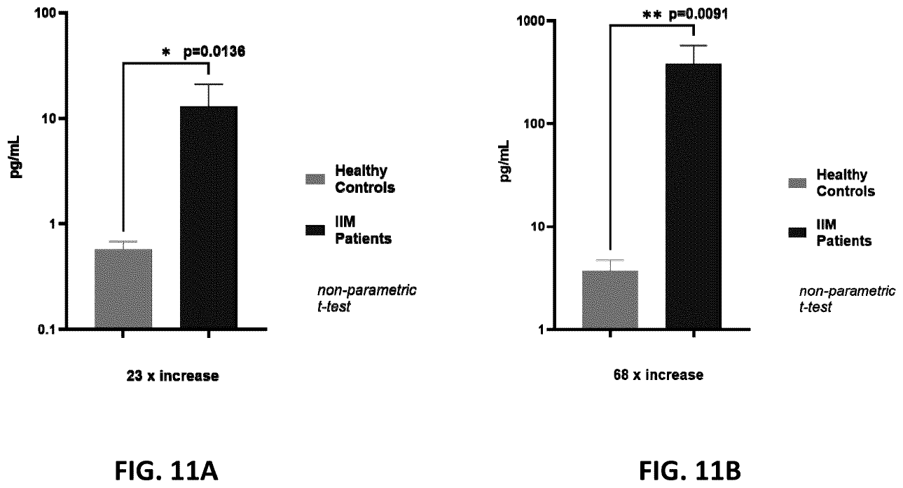

FIGS. 11A-11B provide graphs illustrating significant elevations of IL-12 in patients with idiopathic inflammatory myopathy (IIM) as compared to normal patients without IIM. FIGS. 11A and 11B show the results of IL-12 subunits IL-12p70 and IL-12p40, respectively, measured in patients with IIM and healthy patients. Patients with IIM showed increased amounts of IL-12p70 and IL-12p40 subunits compared to healthy controls. IL-12 is known to be proinflammatory, particularly in promotion of Th1 responses. IL-12p70 is involved in the development of autoimmune disease and plays a role in the pathogenesis of diseases such as psoriasis, and other such diseases. IL-12p70 also interlinks the innate and adaptive immune system via other cytokines such as, for example, IFN-gamma. IL-12p40 also pairs with p19 to form IL-23, another cytokine involved in inflammation and autoimmune diseases. IL-12p40 itself can act as a chemoattractant for macrophages and promote the migration of dendritic cells, both of which are involved in inflammatory responses.

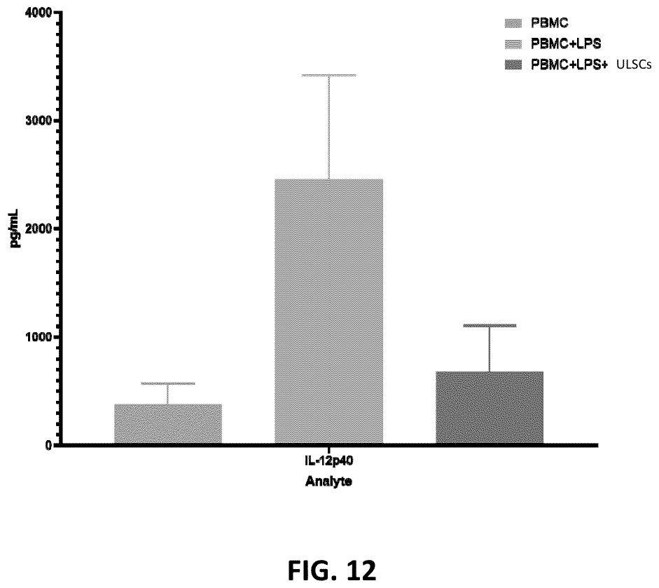

FIG. 12 provides a graph showing the average of IL-12p40 for IIM patients, showing that ULSCs reduce IL-12p40 in patients with IIM, and reduce the inflammatory response of patients' peripheral blood mononuclear cells (PBMCs) when stimulated with LPS. PBMCs alone from patients with IIM have an elevated amount of IL-12p40. When stimulated with LPS beadsP, IL-12p40 was shown to be significantly increased. When mixed with ULSCs, IL-12p40 was shown to be significantly reduced. These data suggest that ULSCs may significantly reduce the amount of IL-12p40 over 60%, and close to baseline.

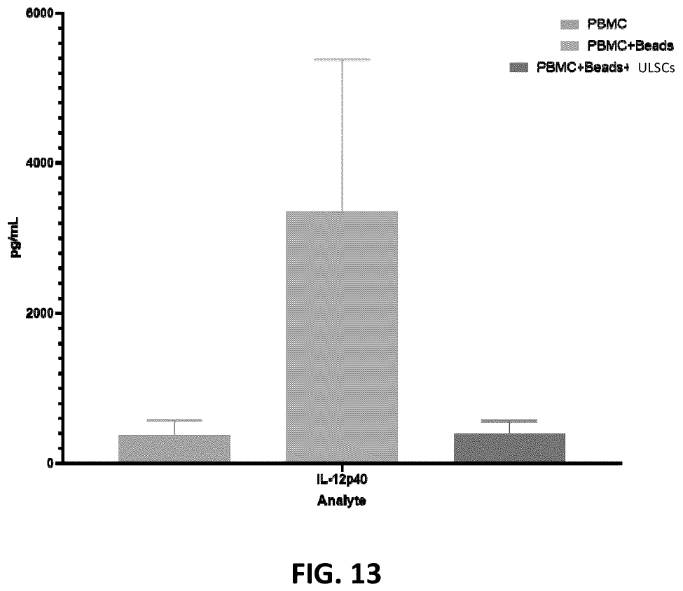

FIG. 13 provides a graph showing the average of IL-12p40 for IIM patients, showing that ULSCs reduce the IL-12p40 inflammatory response of patients' PBMCs when stimulated with inflammatory producing beads. PBMCs alone from patients with IIM have an elevated amount of IL-12p40. When stimulated with beads, IL-12p40 was shown to be significantly increased. When mixed with ULSCs, IL-12p40 was shown to be significantly reduced. Depending on the inflammatory stimulus, these data suggest that ULSCs may significantly reduce the amount of IL-12p40 by over 80% and close to baseline.

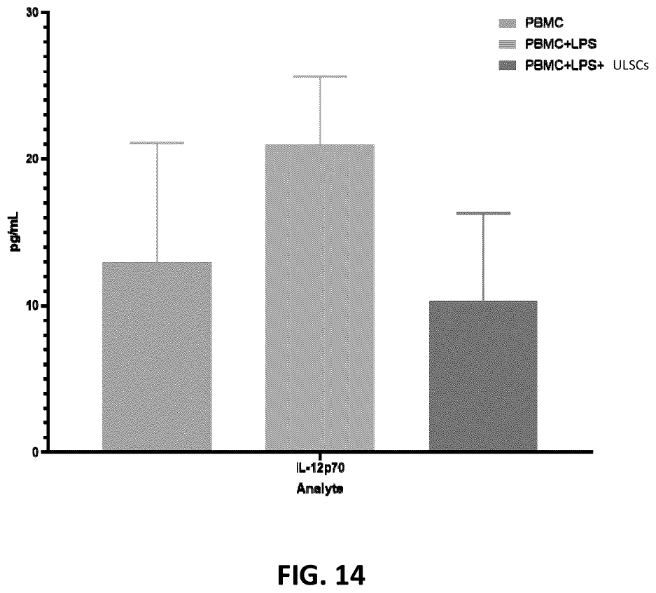

FIG. 14 provides a graph showing the average of IL-12p70 for IIM patients, showing that ULSCs reduce the IL-12p70 inflammatory response of patients' PBMCs when stimulated with LPS. PBMCs alone from patients with IIM have an elevated amount of IL-12p70. When stimulated with LPS beads, IL-12p70 is shown to be significantly increased. When adding stimulating beads and when the beads are mixed with ULSCs, IL-12p70 is shown to be reduced significantly to lower than their baseline. These data suggest that ULSCs may significantly reduce the amount of IL-12p70 by greater than 90% and below baseline, indicating a possible reduction in inflammation.

### MIP-1α Levels in IIM Patients

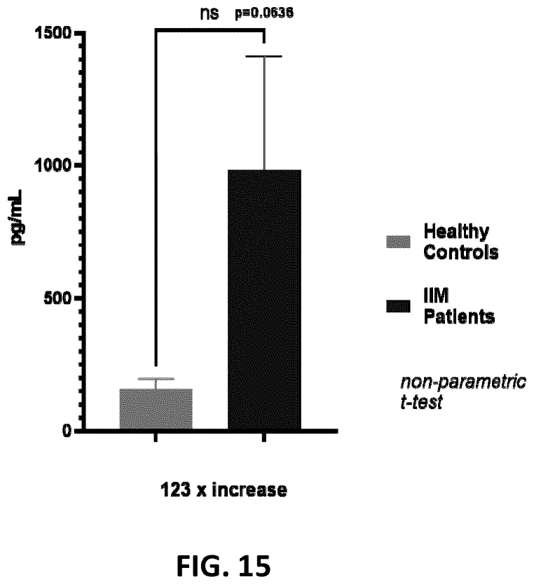

FIG. 15 provides a graph showing significant elevations of the cytokine macrophage inflammatory protein 1α (MIP-1α) in patients with IIM as compared to healthy controls. In IIM, amongst other autoimmune diseases, MIP-1α plays a role in attracting and activating immune cells such as monocytes, and subsets of lymphocytes to sites of inflammation.

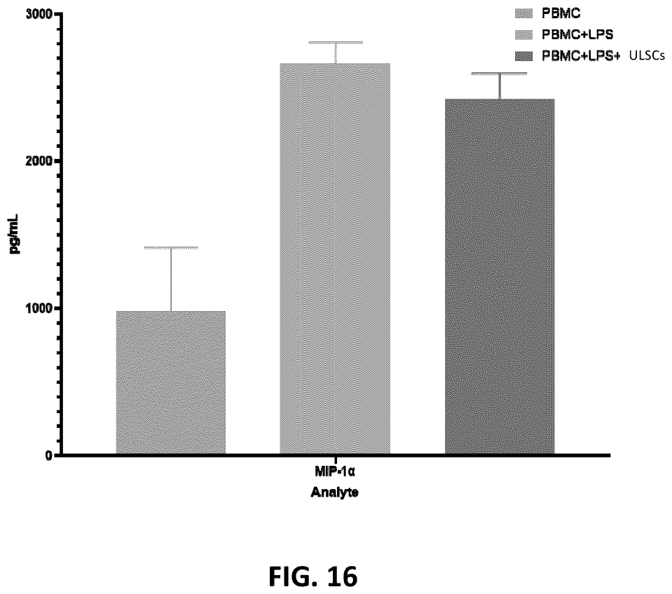

FIG. 16 provides a graph showing the average of MIP-1α for IIM patients, showing that ULSCs reduce the amount of MIP-1α in patients' PBMCs when stimulated with LPS. PBMCs alone from patients with IIM are shown to have an elevated amount of MIP-1α. When stimulated with LPS beads, MIP-1α is shown to be increased significantly. When mixed with ULSCs, MIP-1α is reduced. These data suggest that ULSCs may reduce the amount of MIP-1α which indicates a possible reduction in inflammatory response.

### IL-23 Levels in IIM Patients

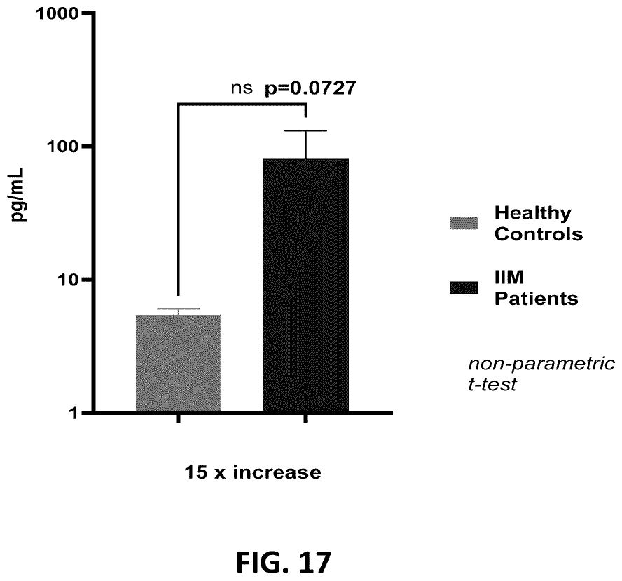

FIG. 17 provides a graph showing IL-23 levels for PBMC baseline, showing that patients with IIM have significantly higher levels of the cytokine IL-23 as compared to healthy controls. IL-23 plays a role in various autoimmune diseases such as inflammatory bowel disease, and also in IIM as suggested by these data. IL-23 promotes the differentiation and activation of TH-12 cells, which are immune cells known to contribute to inflammation in autoimmune conditions.

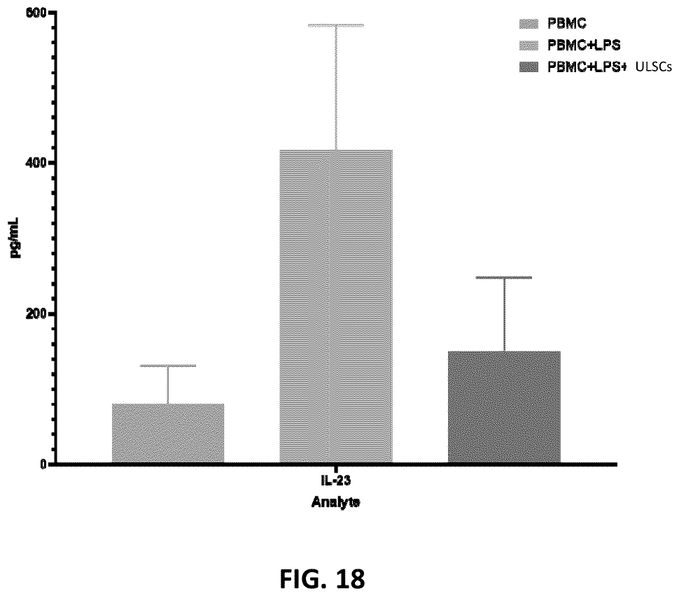

FIG. 18 provides a graph showing the average of IL-23 for IIM patients, showing that, in patients with IIM, PBMCs have significantly higher levels of IL-23, and when stimulated with LPS, IL-23 further increases significantly more. When IIM patients' PBMCs are stimulated with LPS and ULSCs are added, there is a significant reduction in IL-23. These data suggest that ULSCs reduce the inflammatory response mediated by IL-23.

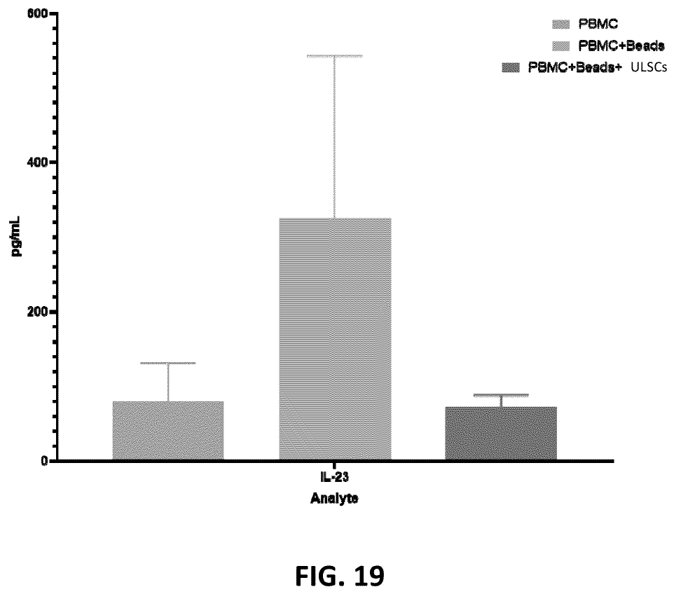

FIG. 19 provides a graph showing the average of IL-23 for IIM patients, showing that, in patients with IIM, PBMCs have significantly higher levels of IL-23, and when stimulated with immune stimulating beads, IL-23 further increases significantly more. When ULSCs are added to a patient's stimulated immune system, there is a significant decrease in the IL-23 mediated inflammatory response. These data suggest a reduction to below baseline inflammatory response in IIM patients.

ULSCs Regulation of Specific Analytes in Patients with Idiopathic Inflammation

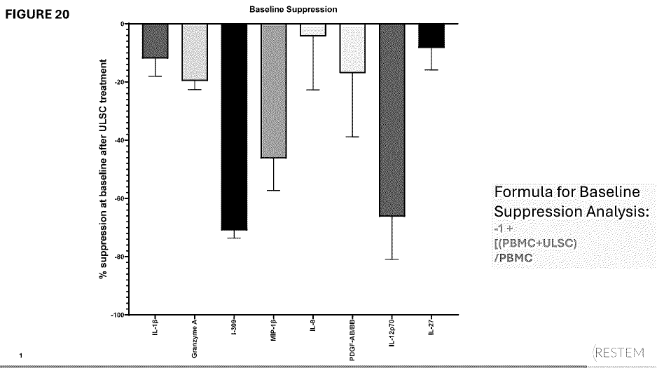

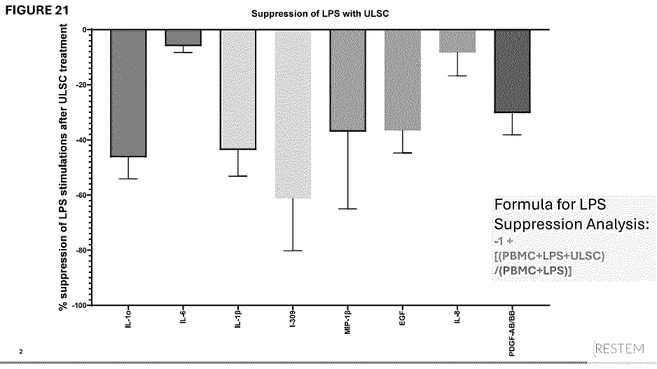

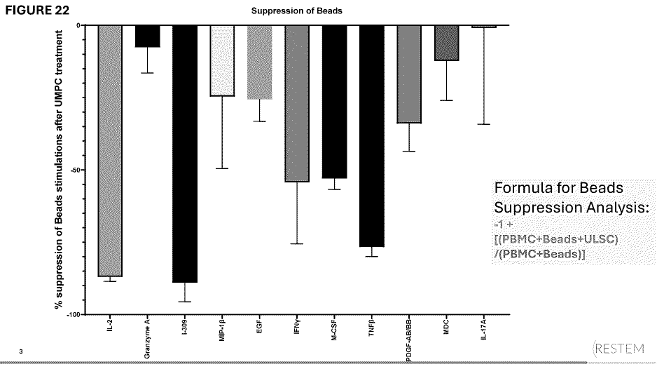

Treatment of PBMC's with ULSCs for 24 hours in vitro results in significant decrease of multiple inflammatory factors including I-309 (CCL1), MIP-1β (CCL4), PDGF-AB/BB, Granzyme A, IL-1β and IL-12p70 (FIG. 20). PBMCs that were inflamed by LPS and CD3/CD28 antibodies also showed significant reductions in some inflammatory factors. After LPS stimulation, the following reductions were observed after UMPC treatment: IL-1α: −46.3%, IL-6: −6%, IL-1β: −43.7%, I-309 (CCL1): −61.3%, MIP-1β (CCL4): −37%, EGF: −36.7%, IL-8: −8.3%, PDGF-AB/BB: −30.3% (FIG. 21). Similarly after CD3/CD28 stimulation, the following factors showed significant reductions after UMPC treatment: IL-2 −87%, Granzyme A −7.7%, I-309 (CCL1) −89%, MIP-1β (CCL4) −24.7%, EGF −25.7%, IFNγ −54.3%, M-CSF −53%, TNFβ −76.7%, PDGF-AB/BB −34%, MDC (CCL22) −12.3%, IL-17A −1% (FIG. 22). Overall, I-309 (CCL1) is consistently and strongly suppressed in all inflammation states, MIP-1β (CCL4) is consistently moderately suppressed across all three conditions, PDGF-AB/BB is consistently and strongly suppressed across all three inflammation states. Granzyme A, IL-8 and IL-1β are moderately suppressed in two out of three conditions

FIG. 21 provides a graph showing suppression of inflammatory factors in the supernatant of PBMCs antagonized by LPS and then treated with ULSCs.

FIG. 22 provides a graph showing suppression of inflammatory factors in the supernatant of PBMCs antagonized by CD3 and CD28 beads and then treated with ULSCs.

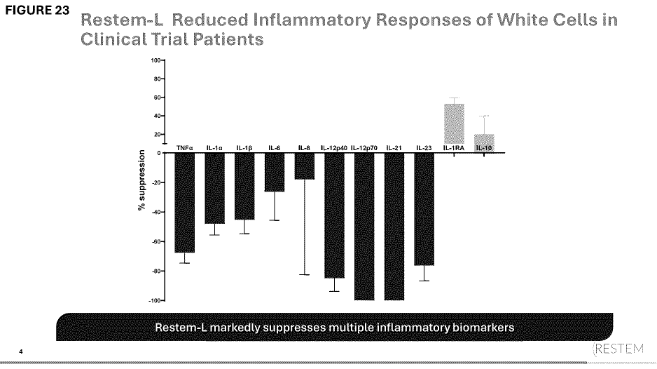

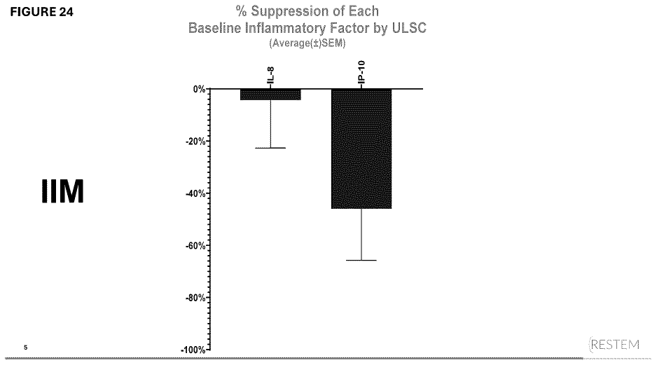

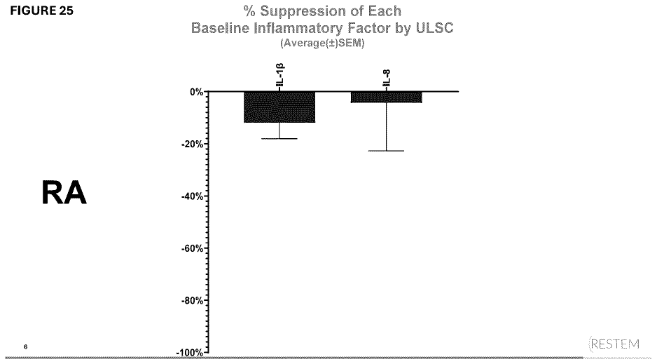

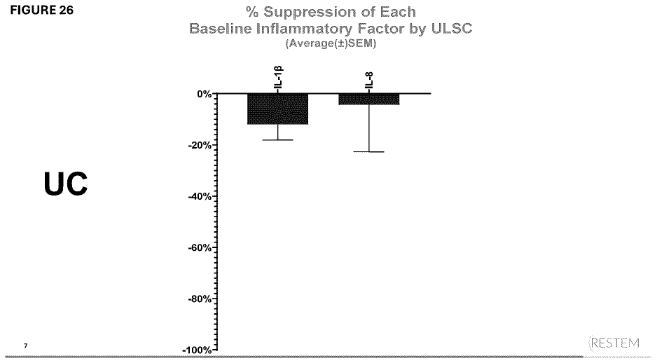

FIG. 23 provides a graph showing reduced inflammatory responses of white cells in clinical trials patients after treatment with ULSCs. Reduction in blood concentrations of inflammatory cytokines was observed across a range of markers specifically IL-1α, IL-1β, IL-6, IL-8, IL-21 and IP-10 while anti-inflammatory cytokines IL-1RA, IL-10 increased. In a patient with IIM, IP-10 concentrations were halved while IL-8 concentrations were reduced potentially by more than 20% (FIG. 24). In a patient with RA, both IL-1β and IL-8 were reduced by potentially about 20% (FIG. 25). In a patient with UC, both IL-1β and IL-8 were reduced by potentially up to about 20% (FIG. 26). Overall the treatment showed effectiveness across a broad range of II conditions at reducing inflammatory factors in human patients.

While certain embodiments have been described, these embodiments have been presented by way of example only, and are not intended to limit the scope of the disclosure. Indeed, the novel methods and systems described herein may be embodied in a variety of other forms, Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics or groups described in conjunction, with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover while operations may be depicted in the drawings or described it he specification in particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount, depending on the desired function or desired result.

The scope of the present disclosure is not intended to be limited by the specific disclosures of preferred embodiments m this section or elsewhere in this specification, and may be defined by claims as presented in this section or elsewhere in this specification or as presented in the future. The language of the claims is to be interpreted broadly based on the language employed m the claims and not limited to the examples described in the present specification or during the prosecution of the application, which examples are to be construed as non-exclusive.

The headings provided herein, if any, are for convenience only and do not necessarily affect the scope or meaning of the devices and methods disclosed herein.

1-9. (canceled)

10. A method of diagnosing idiopathic inflammation in an individual, comprising:
a) Isolating PBMCs from fresh whole blood;
b) Culturing PBMCs in RPMI plus 5% FBS for 24-72 hours at 37° C., 5% CO₂;
c) Collecting conditioned media from said PBMCs;
d) Centrifugation of said conditioned media at 300 g for 7 minutes to isolate supernatant from said PBMCs;
e) Evaluating the level of inflammatory cytokines factors present in said supernatant.

11. The method of claim 10 wherein the inflammatory factors assayed are chosen from the group: TNF-α, IL-12, IL-12p40, IL-12p70, MIP-1α, IL-23, IL-1β, Granzyme A, I-309 (CCL1), MIP-1β (CCL4), IL-8, PDGF-AB/BB, IL-27, IL-1α, IL-6, EGF, IL-2, IFNγ, TNFβ, MDC (CCL22), IL-17A.

12. The method of claim 10 wherein the diagnosis is used as positive selection criterion in personalizing treatment by ULSCs or ULSCs amongst other mesenchymal stem cell therapies and improving the disease state associated with idiopathic inflammation.

13. The method of claim 10, wherein the symptom improvement correlates with a 30-100% increase in IL-10 and IL1-RA in the individual with idiopathic inflammatory myositis.

14. The method of claim 10, wherein the symptom improvement correlates with a decrease in a IL-1β and IL-8 cytokine levels in the individual with rheumatoid arthritis.

15. The method of claim 10, wherein the symptom improvement correlates with a decrease in a IL-1β and IL-8 cytokine levels in the individual with ulcerative colitis.

16-20. (canceled)
